(19) United States
(12) Patent Application Publication
(10) Pub. No.: US 2025/0288930 A1
(43) Pub. Date: Sep. 18, 2025

(54) AN ANTIMICROBIAL HVAC FILTER

(71) Applicant: COPPER CARE WOOD PRESERVATIVES, INC., Humphrey, NE (US)

(72) Inventors: William ABBOTT, Humphrey, NE (US); James JENKINSON, Humphrey, NE (US)

(21) Appl. No.: 18/860,328

(22) PCT Filed: May 1, 2023
(86) PCT No.: PCT/AU2023/050359

§ 371 Date: Oct. 25, 2024

(30) Foreign Application Priority Data
Apr. 29, 2022 (AU) 2022901137

(51) Int. Cl. B01D 46/00; A61L 2/10; A61L 2/238; B01D 39/10; F24F 3/16; F24F 8/108; F24F 8/22; F24F 13/28

(52) U.S. Cl. CPC B01D 46/0028; A61L 2/10; A61L 2/238; B01D 39/10; F24F 3/16; F24F 8/108; F24F 8/22; A61L 2202/11; B01D 2239/0442; B01D 2279/50; B01D 2279/65; F24F 13/28

(57) ABSTRACT

An antimicrobial HVAC filter is designed to be installed in conventional HVAC systems and has conventional filtration material. The filter has antimicrobial enhancement by way of a copper screen and an ultraviolet light source laid flat across the copper screen. The copper provides toxic oligodynamic effect on living cells, algae, molds, spores, fungi, viruses, prokaryotic, and eukaryotic microorganisms. The ultraviolet light source provides germicidal ultraviolet (such as ultraviolet C or UV-C) light to kill or inactivate microorganisms by destroying nucleic acids and disrupting their DNA.

### FIELD OF THE INVENTION

This invention relates generally to a heating, ventilation, and air conditioning (HVAC) filter with antimicrobial enhancements.

### BACKGROUND OF THE INVENTION

Most HVAC systems use a standard 1-inch-thick filter which are installed either in wall-mounted air-return vents (most common in the southern states of America), or at the air handler (more common in the Mid-Atlantic and northern states of America). The air handler is often placed in the basement, near the furnace.

Many filters comprise a cardboard framed board enclosing filtration material therein such as fibreglass, cotton or polyester pleated fabric, high-efficiency particulate air (HEPA) filter material and the like.

Enhanced filters include electrostatic filters which attract dirt and other particles using static electricity, carbon filters which can reduce odours and ultraviolet (UV) light filters which employ UV light to breakdown pathogens of bacteria and viruses using radiation to prevent release of disease-causing particles.

US 2021/0236682 A1 (Willette et al.) 5 Aug. 2021 discloses a dual-effect UV and carbon filter which has a UV LED light array and filter media, such as a fiberglass filter media with a carbon core. The carbon core may be treated with a light activating catalyst.

The photocatalyst composition may be formed with titanium dioxide (TiO₂) in a nano particle form which is doped with dopants such as zinc oxide, zirconium dioxide, nitrogen, silicone, silver (Ag), carbon, iron, or copper to allows the catalyst functionality to work in spectrums above 365 nm, such as at 405 nm. This allows the photocatalyst to work with cheaper visible light sources above 400 nm, such as fluorescent lights, sunlight or LEDS as opposed to more expensive UV based ones.

It is to be understood that, if any prior art information is referred to herein, such reference does not constitute an admission that the information forms part of the common general knowledge in the art, in Australia or any other country.

### SUMMARY OF THE DISCLOSURE

There is provided herein an antimicrobial HVAC filter which can be installed in conventional HVAC systems and which comprises conventional filtration material.

The present filter has antimicrobial enhancement by way of a copper screen and an ultraviolet light source laid flat across the copper screen.

The copper provides toxic oligodynamic effect on living cells, algae, molds, spores, fungi, viruses, prokaryotic, and eukaryotic microorganisms.

The ultraviolet light source provides germicidal ultraviolet (such as ultraviolet C or UV-C) light to kill or inactivate microorganisms by destroying nucleic acids and disrupting their DNA.

Unlike the dual-effect of UV sterilisation and carbon filtration taught by Willette et al., the present antimicrobial filter provides dual-effect by way of UV sterilisation and toxic oligodynamic effect.

Furthermore, unlike Willette et al. which teaches doping of the carbon filter with a photocatalyst to allow the photocatalyst to work with cheaper visible light sources, the copper screen and the ultraviolet light source of the present antimicrobial filter can provide antimicrobial properties independently, including so that the present filter can even provide antimicrobial enhancement even when the ultraviolet light source is not operating.

Furthermore, it is believed that irradiating the copper screen with ultraviolet light source may synergistically enhance the toxic oligodynamic effect thereof.

The ultraviolet light source being laid flat across the copper screen allows the present filter to be inserted into conventional HVAC systems.

The copper screen and the ultraviolet light source may be attached together as a separable unit which can therefore be attached to conventional HVAC filters and detached for cleaning and reuse. The copper screen may be hemmed to prevent fraying at edges thereof. Furthermore, the copper screen may be attached to edges of a conventional HVAC filter board such as with tape. The tape may be reusable tape.

Electrical leads from the ultraviolet light source may extend from one side of the filter to be accessibly attachable to a power supply.

Preferably, the copper screen and the ultraviolet light source are arranged upstream of the filtration material so as to provide antimicrobial effect on microbes accumulating on an upstream surface of the filtration material. Preferably, the filtration material is planar so that the copper screen lies flat there against to maximise contact with microbes accumulated thereon to enhance the toxic oligodynamic effect.

In embodiments, the ultraviolet light source may be configured to direct at least some ultraviolet light directly onto the copper screen as opposed to by way of reflection within the HVAC system. In this regard, the ultraviolet light source may comprise LEDs having lenses which focus light radially or sideways directly onto the copper screen. Alternatively, the ultraviolet light source may have LEDs on free edges of strips which can be compressed flat when inserted into the HVAC system but which lift up once installed so that the ultraviolet light is directed directly onto the copper screen.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a disassembled view of nn antimicrobial HVAC filter in accordance with an embodiment;

FIG. 2 shows an assembled view of the antimicrobial HVAC filter of FIG. 1;

FIG. 3 illustrates installation of the antimicrobial HVAC filter into a conventional HVAC conduit;

FIG. 4 shows a magnified exploded view of an irradiating strip of the filter;

FIG. 5 shows an alternative embodiment of an irradiating strip;

FIG. 6 shows an embodiment of a sideways casting LED lens; and

FIG. 7 shows a top plan view of the ends of FIG. 6.

### DESCRIPTION OF EMBODIMENTS

An antimicrobial HVAC filter 100 comprises 101 filtration material 113. The filtration material 113 may comprise conventional filtration material, such as fibreglass, cotton or polyester pleated fabric, high-efficiency particulate air (HEPA) filter material. The filtration material 113 may be enclosed in a conventional cardboard framed board 101. In embodiments, the board 101 may be approximately 1 inch thick as to fit conventional HVAC systems.

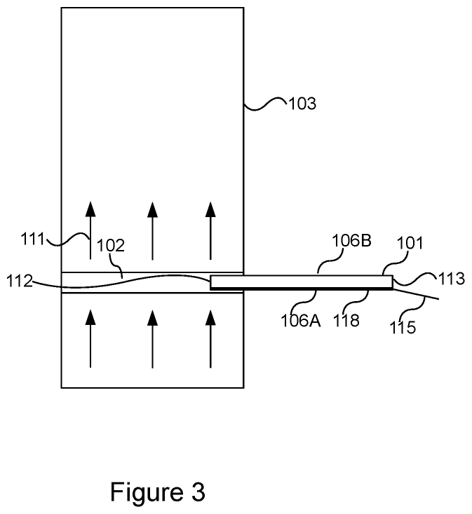

As shown in FIG. 3, the filter 100 may be configured to be insertable sideways into a rack or slot 102 of an HVAC air handler conduit 103. Alternatively, the filter 100 may be configured to be insertable in a wall-mounted air-return vent.

The filter 100 comprises a copper screen 118. The copper screen 118 preferably has a fine mesh of copper wire to enhance surface contact with pathogens.

The filter 100 further comprises an ultraviolet light source laid flat across the copper screen 118.

The copper screen 118 provides toxic oligodynamic effect on living cells, algae, molds, spores, fungi, viruses, prokaryotic, and eukaryotic microorganisms. The ultraviolet light source provides germicidal ultraviolet light to kill or inactivate microorganisms. Preferably, the ultraviolet light is ultraviolet C or UV-C.

The ultraviolet light source being laid flat across the copper screen 118 allows the filter 100 to be inserted into conventional HVAC systems such as is shown in FIG. 3.

In the embodiment shown, the ultraviolet light source comprises at least one strip 104 of ultraviolet light emitting LEDs 105 which are laid flat across a surface 106 of the copper screen 118. The strips 104 lie flat across the surface 106 of the copper screen 118, thereby not affecting the thickness or cross-sectional low profile of the board 101. As such, the filter 100 comprising the laid-flat strips 104 may be yet inserted into relatively confined HVAC racks or slots 102 in the manner shown in FIG. 3.

The copper screen 118 and the ultraviolet light source may be attached together as a separable unit which can therefore be releasably attachable to the board 101. As such the copper screen 118 and ultraviolet light source unit may be attached to the board 101, such as at edges thereof, such as by using tape. When the filtration material 113 requires replacement, the separable unit may be detached from the board 101 for cleaning and reuse by reattachment to new filtration material 113.

In alternative embodiments, the copper screen 118 and the ultraviolet light source are not separable from the board 101 and which are disposed of with the board 101.

Edges of the copper screen 118 may be hemmed to prevent fraying. Hemming may be provided by way of folding back and compressing edges of the copper screen 118 or crimping the edges of the copper screen between metallic or plastic crimping material.

With reference to FIG. 3, when installed in the conduit 103, the filter 100 may define an upstream side 106A and a downstream side 106B. Preferably, the copper screen 118 and the ultraviolet light source are on the upstream side 106A upstream side of the filtration material 113 to thereby provide antimicrobial effect on pathogens collected on the upstream surface 106A of the filtration material 113.

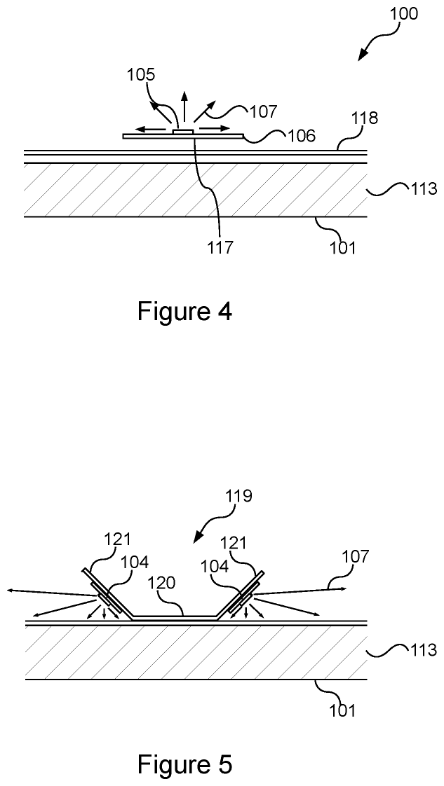

The strips 113 may be attached flat to an outer surface of the copper screen 118. As is shown in FIG. 4, the LEDs 105 thereof may predominantly emit UV light 107 away from the copper screen 118 which can reflect back against internals of the HVAC system onto the copper screen 118 and the filtration material 113.

The LEDs 105 may be configured so that at least some of the UV light 107 is directed directly onto the copper screen 118. For example, the LEDs 105 may be configured to emit the ultraviolet light 107 through an arc of 180° or more, so that at least some of the ultraviolet light is emitted sideways directly onto the copper screen 118.

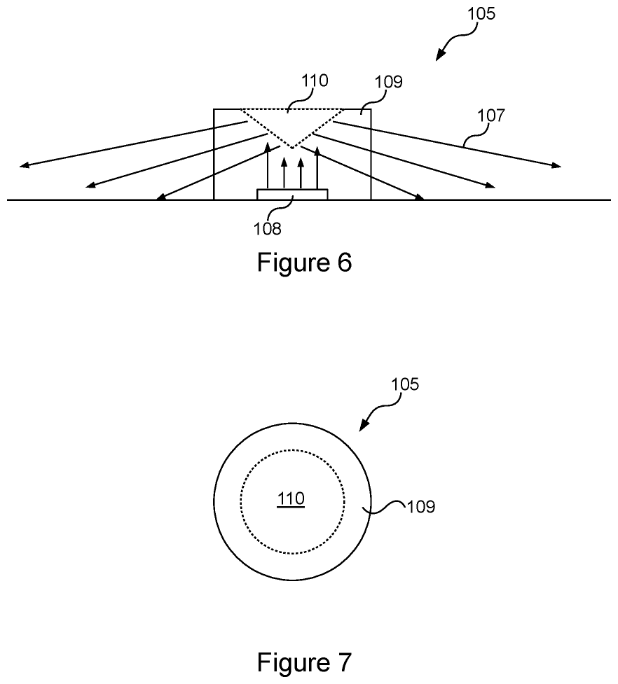

In the embodiment shown in FIGS. 6 and 7, each LED 105 may comprise an integrally formed lens 109 above a diode 108 thereof. As such, ultraviolet light 107 propagating substantially perpendicularly from the diode 108 may be reflected sideways by a substantially conical concavity 110 to thereby increase the radiation intensity across the surface 106 of the copper screen 107. Whereas the concavity 110 may be generally circular so as to radially spread the radiation around the LED 105, in embodiments, the concavity 110 may be substantially symmetric with respect to a longitudinal axis of the strip 104 to thereby spread light sideways with respect to the strip 104.

In embodiments, the ultraviolet light source is configurable in a flush fit configuration wherein the LEDs 105 thereof are flattened against the copper screen 118 to aid insertion of the filter 100. However, once installed, the LEDs 105 may raise away from the copper screen 118 to emit light directly onto the copper screen 118.

Specifically, as is shown in FIG. 5, a flexible channel strip 119 may be employed which comprises a central section 120 which is adhered to the surface of the coper screen 107 and which defines free edges 121 which are biased upwardly. The LEDs 105 may be installed on under surfaces of these free edges 121 to direct UV light directly onto the copper screen 118 thereunder. More specifically, LED strips 104 may be installed under the respective free edges 121.

As such, when the filter 100 is inserted into the slot, the edges 121 flatten against the copper screen but, once free inside the conduit 103, the free edges 121 may pop up in the manner shown, thereby positioning the strips 104 to cast ultraviolet light directly onto the surface of the copper screen 118.

The LEDs 105 preferably emit ultraviolet light. The ultraviolet light is further preferably UV-C radiation. Yet further, the radiation preferably peaks at approximately 265 nm.

As shown in FIG. 3, the filter board 101 may define a distal edge 112 which is inserted first into the slot 102 and a proximal edge 113. The electrical power leads 115 may protrude from the proximal edge 113 for convenient connections to a power supply once the filter 100 is installed.

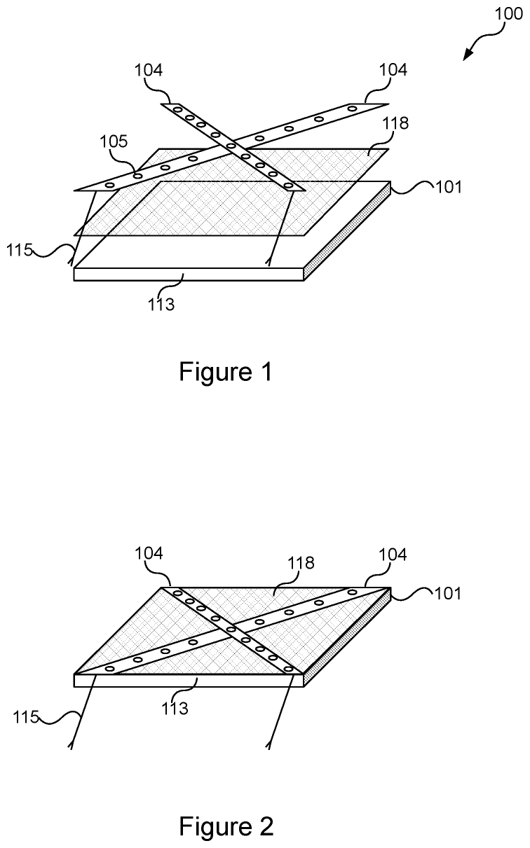

The ultraviolet light source is preferably configured so that intensity of ultraviolet light emitted by the ultraviolet light source peaks towards a centre of the filter 100. As shown in FIG. 1, the ultraviolet light source may comprise two strips 104 of LEDs 105 attached flat across a surface of the copper screen 118 and wherein the strips 104 cross over near the centre of the filter 100. The strips 104 may run between diagonal corners of the filter board 101 as shown to maximise the length of each strip 104.

The apertures of the copper screen 118 may be carefully sized so as to not be unduly small to clog up with dust, debris and the like whilst yet being able to maximise surface contact area for the toxic oligodynamic effect.

The antimicrobial HVAC filter 100 may be sold with the strips 105 preinstalled on the filter board 101. Alternatively, a separable unit comprising the copper screen 118 and the ultraviolet light source may be attached to a conventional filter board 101.

The filter board 101 may be sized and shaped for installation in conventional HVAC systems. As such, the user may remove an existing filter from the conduit 103 and insert the antimicrobial HVAC filter 101 into the slot 102 with the distal edge 112 first.

The low-profile nature of the laid-flat strips 104 does not interfere with the installation of the antimicrobial HVAC filter 100 into the conventional HVAC conduit 103 slot 102.

As alluded to above, the user may install the antimicrobial HVAC filter 100 with the copper screen 118 and the ultraviolet light source facing the air stream 111 on the upstream side 106A. The user may then electrically connect the electrical leads 115 extending from the filter 100 from the proximal edge 113 to a power supply. In embodiments, the power supply only supplies power to the antimicrobial HVAC filter 100 when the HVAC system is running. As such, during normal operation of the HVAC system, the filter board 101 collects dust and pathogens on the upstream surface 106A thereof which are treated by the ultraviolet light source and the copper screen 118.

The ultraviolet light source and the copper screen 118 provide a combined UV sterilisation and toxic oligodynamic antimicrobial effect. The filter 100 may also provide antimicrobial effect when the ultraviolet light source is not operating wherein the copper screen 118 provides toxic oligodynamic antimicrobial effect alone.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. An antimicrobial HVAC filter comprising filtration material and being configured to insert into an HVAC conduit in use, wherein filter comprises a copper screen and an ultraviolet light source laid flat across the copper screen.

2. The filter as claimed in claim 1, wherein the copper screen and the ultraviolet light source are upstream of the filtration material.

3. The filter as claimed in claim 1, wherein the filtration material has a planar surface and wherein the copper screen is laid flat against the planar surface.

4. The filter as claimed in claim 1, wherein the copper screen and the ultraviolet light source are attached together as a unit separable from a filter board comprising the filtration material.

5. The filter as claimed in claim 4, wherein the copper screen is attached to the board at edges of the copper screen.

6. The filter as claimed in claim 5, wherein the copper screen is attached to edges of the board with tape.

7. The filter as claimed in claim 4, wherein edges of the copper screen are hemmed.

8. The filter as claimed in claim 1, wherein the ultraviolet light source comprises strips of LEDs attached flat across a surface of copper screen.

9. The filter as claimed in claim 8, wherein the strips have adhesive backings which adhere to the surface copper screen.

10. The filter as claimed in claim 8, wherein the strips are attached flat across an outer surface of the copper screen and wherein the LEDs thereof predominantly emit the light away from the copper screen.

11. The filter as claimed in claim 8, wherein the ultraviolet light source is configured to direct at least some ultraviolet light directly only the copper screen.

12. The filter as claimed in claim 11, wherein the LEDs emit light through an arc of approximately 180° or more.

13. The filter as claimed in claim 11, wherein the LEDs have lenses which direct the light sideways across the copper screen.

14. The filter as claimed in claim 13, wherein the lenses each form an upper concavity which reflects the light sideways.

15. The filter as claimed in claim 14, wherein the upper concavity is conical.

16. The filter as claimed in claim 14, wherein the upper concavity is symmetric with respect to a longitudinal axis of the irradiating strip.

17. The filter as claimed in claim 1, wherein the ultraviolet light source is configurable in a flattened configuration wherein LEDs thereof are flattened against the copper screen and an inserted configuration wherein the LEDs are raised away from the screen and emit light directly onto the screen.

18. The filter as claimed in claim 17, wherein the ultraviolet light source comprises a flexible channel strip comprising a central section which is adhered to the surface of the copper screen and a free edge which is biased upwardly and wherein the LEDs are adhered to undersurfaces of the free edge.

19. The filter as claimed in claim 18, wherein the strip comprises free edges on both sides thereof, each free edge having respective LEDs.

20. The filter as claimed in claim 1, wherein the ultraviolet light source emits ultraviolet radiation peaking at approximately 265 nm.

21. The filter as claimed in claim 1, wherein the ultraviolet light source emit UV-C radiation.

22. The filter as claimed in claim 1, wherein the ultraviolet light source is configured so that intensity of light emitted by the ultraviolet light source peaks towards a centre of the filter.

23. The filter as claimed in claim 22, wherein the ultraviolet light source comprises two strips of LEDs attached flat across a surface copper screen and wherein the strips cross over at the centre of the filter.

24. The filter as claimed in claim 23, wherein the strips run between diagonal corners of the filter.

25. The filter as claimed in claim 1, wherein the board defines a distal insertion edge and a proximal edge and wherein electrical leads for the ultraviolet light source extend from the proximal edge.

26. The method involving the antimicrobial HVAC filter as claimed in claim 1, the method comprising inserting the antimicrobial HVAC filter into an HVAC conduit filter rack and powering the ultraviolet light source so that the UV light source and the copper screen provide combined UV sterilisation and toxic oligodynamic antimicrobial effect.

27. The filter as claimed in claim 26, wherein the filter is inserted so that the copper screen and ultraviolet light source face upstream.

28. A method involving the antimicrobial HVAC filter as claimed in claim 1, the method comprising inserting the antimicrobial HVAC filter into an HVAC conduit filter rack and either:
powering the ultraviolet light source so that the UV light source and the copper screen provide combined UV sterilisation and toxic oligodynamic antimicrobial effect; and
not powering the ultraviolet light source so that the copper screen provides toxic oligodynamic antimicrobial effect alone.
